(19) United States
(12) Patent Application Publication
(10) Pub. No.: US 2024/0417816 A1
(43) Pub. Date: Dec. 19, 2024

(54) METHODS FOR GENETIC ANALYSIS OF TEXTILES COMPRISING G. BARBADENSE

(71) Applicant: Applied DNA Sciences, Inc., Stony Brook, NY (US)

(72) Inventor: Ling DONG, South Setauket, NY (US)

(73) Assignee: Applied DNA Sciences, Inc., Stony Brook, NY (US)

(21) Appl. No.: 18/703,558

(22) PCT Filed: Oct. 21, 2022
(86) PCT No.: PCT/US2022/047411

§ 371 Date: Apr. 22, 2024

Related U.S. Application Data
(60) Provisional application No. 63/270,346, filed on Oct. 21, 2021.

(51) Int. Cl. C12Q 1/6895

(52) U.S. Cl. CPC C12Q 1/6895; C12Q 2600/156

(57) ABSTRACT

The present invention provides a method for determining whether a mature cotton fiber includes Giza 94 (G94) variety of G. barbadense. The method comprises providing a mature cotton fiber from a manufactured article; extracting DNA from the mature cotton fiber; amplifying a portion of said DNA with a PCR-based technique using a primer set comprising SEQ ID NO: 1 and SEQ ID NO: 2, thereby producing one or more amplicons, generating a variable amplicon length polymorphism profile (VALP) from the amplicons, and comparing the generated VALP with a G94 variable amplicon length polymorphism profile, thereby determining whether the mature cotton fiber incudes G94.

### FIELD OF THE INVENTION

This invention pertains to methods for analyzing mature cotton fibers for the detection of genetic variations among a variety of cotton cultivars of a cotton species, particularly, cultivars of the cotton species Gossypium barbadense.

### BACKGROUND OF THE INVENTION

In general, there are two major types of cotton species being cultivated throughout the world, namely Gossypium barbadense and Gossypium hirsutum. These two species are from the genus Gossypium, which comprises at least 40 different cotton species. Gossypium barbadense (sea island cotton) and Gossypium hirsutum (upland cotton) are allotetraploids and are known as New World cotton or “American” cotton. There are striking differences in the physical characteristics of the cotton fibers produced by G. barbadense compared to the cotton fibers produced by G. hirsutum. G. barbadense produces longer cotton fibers than most other cotton species and these fibers are usually called “extra long staple” (ELS) fibers, while those of G. hirsutum are shorter and are called or defined as “upland” fibers. Textiles made of ELS fibers are considered to be of higher quality compared to textiles made with shorter fibers, like those produced by G. hirsutum cotton plants. Thus, textiles produced from ELS cotton fibers are considered more valuable in the textile marketplace.

Many regions around the world produce ELS cottons with distinct fiber qualities, such as Egyptian Pima and Indian Pima. Despite the common ancestry, over time, isolation and/or cross breeding of a cotton species has created subtle but unique genetic variations in cultivars from different regions. Even though these cotton types belong to the same species, G. barbadense, ELS from certain regions, such as American Pima, have superb fiber qualities compared to G. barbadense grown in other regions of the world and are heavily promoted and highly sought after by textile manufacturers. Thus, certain ELS cottons from a particular geographical region are more valuable than others. All ELS cotton cultivars are within the G. barbadense species, and there is really no physical measurement(s) which can readily distinguish between various ELS cotton cultivars produced from different geographical regions.

Unfortunately, once cotton fibers are processed and made into yarn and/or fabrics, there is no reliable method to determine the origin or cultivar of the fibers utilized to produce the yarn or textile(s). Forging clothing or producing knock-off textile items is a serious problem for the textile industry, costing manufacturers and retail stores millions and perhaps billions of dollars annually, in the United States alone. Some manufacturers are using inferior quality cotton or other cheaper ELS cotton to cut costs. This is not necessarily equivalent to making fake products, but this practice still impacts not only the brand owner but also the cotton producers. Being able to identify the cultivar of a particular species of cotton utilized in a textile item would not only be a way to authenticate an item as legitimate and being made with the type of cotton specified by the owners, but would also enable the detection of forged or counterfeit textile products.

There have been many studies trying to manipulate cotton genes for fiber quality improvement (U.S. Pat. Nos. 6,169,174; 7,060,874; and 6,995,256), enhanced pesticide toxin production (U.S. Pat. Nos. 6,686,149; 6,140,075; and 6,057,370), and herbicide resistance (U.S. Pat. No. 7,223,906). There have also been studies investigating the genetic polymorphism of various cotton species using PCR-based markers (J. Applied Sci. Res. 3 (10) 1156-1169, 2007). A variety of studies have demonstrated that DNA heterogeneity within strains of similar species can arise both in intragenic (inside) and intergenic (outside) genetic coding regions, and that polymorphism at the level of DNA yields a remarkably specific signature for individuals, strains and species. However, no success has been found in the categorization of cotton cultivars using genetic markers on mature cotton fibers mainly because of the lack efficient primers to amplify fragmented DNA in mature cotton fibers.

### SUMMARY OF THE INVENTION

In one aspect, the present invention provides a method for determining whether a mature cotton fiber includes Giza 94 (G94) variety of G. barbadense. The method comprising: providing a mature cotton fiber from a manufactured article; extracting DNA from the mature cotton fiber; amplifying a portion of said DNA with a PCR-based technique using a primer set comprising SEQ ID NO: 1 and SEQ ID NO: 2, thereby producing one or more amplicons, generating a variable amplicon length polymorphism profile (VALP) from the amplicons, and comparing the generated VALP with a G94 variable amplicon length polymorphism profile, thereby determining whether the mature cotton fiber incudes G94. The extracted DNA may comprise nuclear DNA, mitochondrial DNA and/or chloroplast DNA. In one embodiment, the generated VALP is obtained by capillary electrophoresis. In one embodiment, the G94 variable amplicon length polymorphism profile comprises peaks in capillary electrophoresis traces at 115 bp, 121 bp, 126 bp and 128 bp. In one embodiment, the generated VALP is obtained by gel electrophoresis. In one embodiment, the method further comprises quantifying the one or more of the amplicons by real-time PCR. In one embodiment, the method further comprises analyzing an additional detectable physical characteristic. In one embodiment, the additional detectable physical characteristic comprises a difference in nucleotide composition. In one embodiment, analyzing an additional detectable physical characteristic comprises the use of a hybridizing probe specific for the target sequence of G94. In one embodiment, the manufactured article comprises raw mature cotton, ginned mature cotton, yarn blended with mature cotton and/or fabric blended with mature cotton. Examples of manufactured articles include a garment or textile. Examples of textiles are those of yarn, fabric, ginned cotton, an artwork canvas and an anthropological textile. In another aspect, the present invention includes a method for verifying authenticity of the variety of G. barbadense of a mature cotton fiber as including a fiber from G94, wherein the mature cotton fiber is from a manufactured article. The method comprises providing a mature cotton fiber from the manufactured article; extracting DNA from the mature cotton fiber; amplifying a portion of said DNA with a PCR-based technique using a primer set consisting of SEQ ID NO: 1 and SEQ ID NO: 2, thereby producing one or more amplicons, generating a variable amplicon length polymorphism profile (VALP) from the amplicons, and comparing the generated VALP with a G94 variable amplicon length polymorphism profile, thereby verifying the authenticity of the mature cotton fiber from the manufactured article as including a fiber from G94.

### BRIEF DESCRIPTION OF THE FIGURES

FIG. 1 is a PCR/CE assay showing an extra peak at ˜128 bp in Giza94 (lower panel) that does not exist in Giza86 (upper panel) or in US Pima.

FIG. 2 is a diagram showing the comparison of a genetic variable region of two different cotton cultivars.

FIG. 3 is a flow chart of one embodiment of the methods for identifying the cotton cultivar of mature cotton fibers.

### DETAILED DESCRIPTION OF THE INVENTION

Unless otherwise stated, the following terms used in this application, including the specification and claims, have the definitions given below. It must be noted that, as used in the specification and the appended claims, the singular forms “a”, “an,” and “the” include plural referents unless the context clearly dictates otherwise.

The term “ELS” means “extra long staple” cotton fibers. For example, those fibers produced from G. barbadense are ELS cotton fibers.

The term “cultivar” means a cultivated plant that has been selected and given a unique name because it has desirable characteristics that distinguish it from otherwise similar plants of the same species. Cultivar may also mean “sub-species.”

The term “genotype” means the unique genetic characteristics of a particular species or cultivar. Genotyping is considered the detection of a particular genotype of a species or sub-species.

The term “Upland fiber” defines cotton fibers which are shorter than ELS cotton fibers. For example, upland fibers are produced from G. hirsutum.

The term “genomic DNA” means the full complement of DNA contained in the genome of a cell or organism. This includes nuclear DNA as well as DNA stored within organelles, such as the mitochondrial genome (mDNA) and the chloroplast genome (cpDNA).

The term “variable region” means a genetic region of similar cotton species which has sequence variation between cultivars or species. The variation may be for example, a difference in sequence length within a genetic region, or single nucleotide changes within in specific genetic region.

The term “variable length polymorphism” means a variety of sequence lengths have been identified or detected for a targeted region across various cotton species or sub-species, such as cultivars.

The term “primer” means a nucleotide with a specific nucleotide sequence which is sufficiently complementary to a particular sequence of a target DNA molecule, such that the primer specifically hybridizes to the target DNA molecule.

The term “probe” refers to a binding component which binds preferentially to one or more targets (e.g., antigenic epitopes, polynucleotide sequences, macromolecular receptors) with an affinity sufficient to permit discrimination of labeled probe bound to target from nonspecifically bound labeled probe (i.e., background).

The term “probe polynucleotide” means a polynucleotide that specifically hybridizes to a predetermined target polynucleotide.

The term “oligomer” refers to a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The term “PCR” refers to polymerase chain reaction. This refers to any technology where a nucleotide is amplified via temperature cycling techniques in the presence of a nucleotide polymerase, preferably a DNA polymerase. This includes but is not limited to real-time PCR technology, reverse transcriptase-PCR, touchdown PCR and standard PCR methods.

The term “nucleic acid” means a polymer composed of nucleotides, e.g. deoxyribonucleotides or ribonucleotides, or compounds produced synthetically which can hybridize with naturally occurring nucleic acids in a sequence specific manner analogous to that of two naturally occurring nucleic acids, i.e., can participate in hybridization reactions, e.g., cooperative interactions through Pi electron stacking and hydrogen bonds, such as Watson-Crick base pairing interactions, Wobble interactions, etc.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” or “nucleotide” refer to single or double stranded polymer composed of nucleotide monomers of generally greater than 50 nucleotides in length.

The term “monomer” as used herein refers to a chemical entity that can be covalently linked to one or more other such entities to form an oligomer. Examples of “monomers” include nucleotides, amino acids, saccharides, peptides, and the like.

The term “identifiable sequence” or “detectable sequence” means a nucleotide sequence which can be detected by hybridization and/or PCR technology by a primer or probe designed for specific interaction with the target nucleotide sequence to be identified. The interaction of the target nucleotide sequence with the specific probe or primer can be detected by optical and/or visual means to determine the presence of the target nucleotide sequence.

In general, the term “textile” may be used to refer to fibers, yarns, or fabrics. More particularly, the term “textile” as used herein refers to raw cotton, ginned cotton, cotton fibers, cotton yarns, cotton fabrics, yarn that is blended with cotton, fabric that is blended with cotton, or any combination thereof.

The term “mature cotton fiber” as used herein refers to a cotton fiber having a lumen wall that separates the secondary wall (consisting of cellulose) from the lumen that has naturally collapsed. The lumen is the hollow canal that runs the length of the fiber and is filled with living protoplast during the growth period; after the fiber matures and the boll opens the protoplast dries up and the lumen will naturally collapse and leave a large central void in each fiber.

The present invention includes methods for identifying and verifying a cultivar of a particular cotton species using mature cotton fibers. Being able to determine the particular cultivar of a cotton shipment or textile allows for the quality of the cotton to be verified, which aids in protecting the reputation of high end textile brands.

In particular, the methods relate to verifying the type/cultivar of cotton used in the manufacturing of an article comprising cotton. There are four major cotton species utilized in textiles around the globe, and they are G. barbadense, G. hirsutum, G. arboreum, and G. herbaceum. Each one of these cotton species has a plurality of cultivars, and the cultivars from a particular species can vary considerably in quality. The present methods allow for the identification and distinction of one cultivar from other cultivars of the same species. The present methods allow for cultivar genotyping of a wide range of cotton species.

Most of the cotton textiles produced are made from two cotton species, G. barbadense and G. hirsutum, and both of these species comprise a plurality of cultivars, each cultivar having unique genotypic and phenotypic properties from one another. Methods are provided for identifying “Extra Long Staple” (ELS) cottons or cultivars of G. barbadense grown from different geographical regions of the world.

The methods allow a user to genetically identify textiles made of cotton fibers from a particular cotton cultivar. The process involves extracting DNA from mature cotton fibers or textiles and amplifying DNA using specific primers and the Polymerase Chain Reaction (PCR) method. Specific primers targeting regions exhibiting sequence variation or sequence polymorphism among Gossypium cultivars were used to distinguish various types of cultivars.

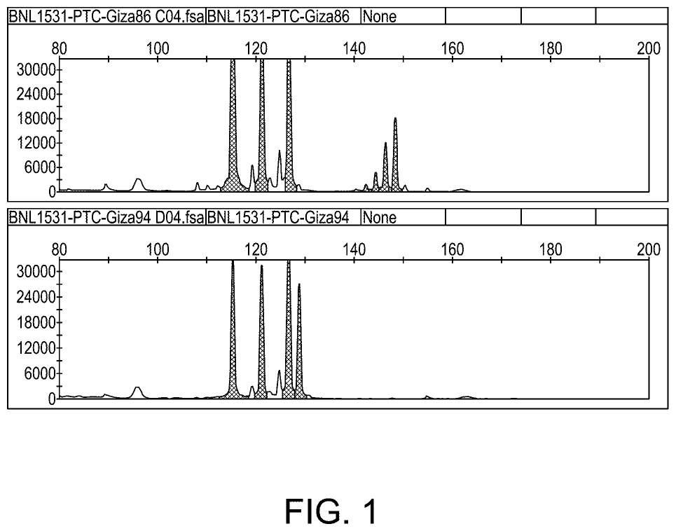

There are subtle genetic variations in the genetic makeup of the G. barbadense/ELS cultivars. The methods provide genetic tests which definitively identify fibers and textiles as being made from a particular ELS cultivar. The methods utilize at least one variable sequence region found within the cotton genomes of interest, which allows the cotton cultivars to be distinguished from one another. In general, to identify cotton fibers originating from a particular cultivar, endogenous genetic markers based on the genetic variation between the cultivars of interest were utilized and are depicted in FIG. 1.

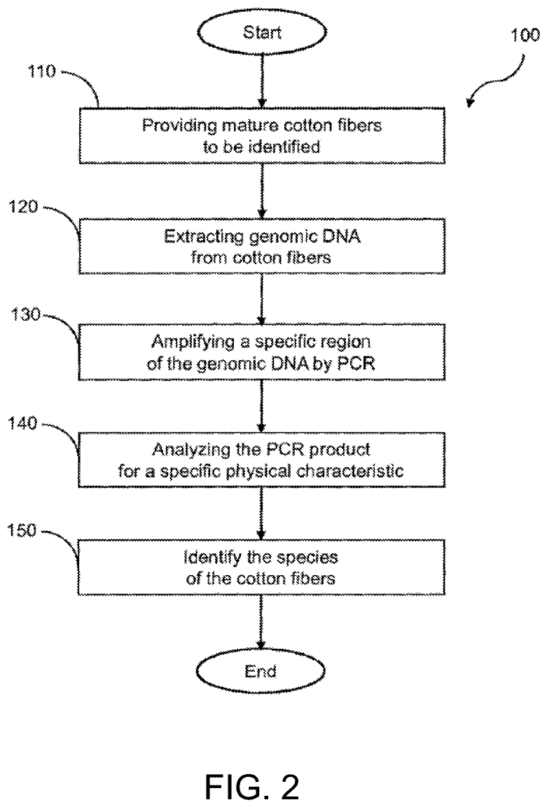

FIG. 2 is a graphical representation showing the comparison of a genetic variable region of Extra Long Staple (ELS) cotton cultivars. A variable region can differ in either DNA length or DNA sequence, while the non-variable regions have identical DNA sequences between the cultivars being compared. Thus, the variable region can be utilized as an endogenous marker to distinguish between ELS cotton cultivars. By targeting regions of the DNA genome(s), which have sequence variations such as the region depicted in FIG. 1, the mature cotton fibers can be identified as a specific cotton cultivar by the length or size of the target sequence(s). To genotype ELS cotton cultivars, several variable sequence regions within the cotton genome which identify different ELS cottons were utilized. The method allows for cotton sequence-specific primers associated with simple sequence repeats (SSRs) for ELS cultivar differentiation. This allows the use of SSR primers to detect endogenous markers with varying DNA length polymorphisms between the different cultivars.

While sequence length is the physical characteristic targeted by the endogenous marker shown in FIG. 1, the physical characteristic used to identify the cotton fibers as a particular ELS cultivar could be differences in site specific mutations which can be detected by specific probes designed for each cotton cultivar of interest.

Generally, ELS sequence-specific primers associated with simple sequence repeats (SSRs) are used to amplify DNA from mature cotton fibers or textiles and DNA variable length polymorphism (genotyping) was used to distinguish different ELS cultivars. When multiple primers are used, they create a powerful genotyping tool that is capable of discriminating thousands of ELS cultivars. The process involves extracting DNA from mature cotton fibers or textiles and amplifying DNA using PCR methods. Specific primer sets targeting regions exhibiting sequence variation or sequence polymorphism among different ELS are used to identify individual ELS cultivars. PCR results were analyzed by capillary electrophoresis for DNA length polymorphism, and results from multiple primers sets created a unique genotype for individual ELS cultivars.

By testing all available ELS cultivars using the methods described herein, an ELS identity gene bank can be established and each ELS cultivar can be associated with unique DNA genotype.

DNA fragment size was determined by an internal size standard using best-fit second order curve interpolation; therefore, DNA sequence lengths from different runs are considered to have the same genotype if the variation is within one base pair. Each SSR primer set generates multiple DNA sequence lengths for each ELS cultivar. The multiple DNA length products produced in the cultivars is due to ELS cotton species being tetraploid, i.e. having a possibility of four different chromosomal sets. The methods demonstrate that a combination of primer sets can discriminate between thousands of different cultivars.

In other embodiments, SNP or haplotyping are utilized to distinguish different cotton cultivars. Primers are designed to amplify DNA fragments containing SNP/haplotypes and then the amplified DNA fragments are analyzed by sequencing. SNP analysis kits are available, such as the TaqMan™ kit by ABI, or SNP analysis can be carried out by a synthesis method, such as Pyrosequencing™.

FIG. 2 is a flow chart illustrating one embodiment of a method 100 for identifying the cultivars of mature cotton fibers. At event 110, mature cotton fibers are collected and/or obtained from a cotton source. The cotton source may be raw cotton fibers from a specific geographical location, from a cotton textile, from a cotton textile article or garment, or from a source were the cotton cultivar is already known. The cotton fibers may also be from anthropological textiles as well as fibers from artwork canvases or any other article where knowing the cotton cultivar utilized for an article would be advantageous in verifying or authenticating the item. The methods allow for verification of articles in a manner that helps prevent forgers and counterfeiters from substituting false or counterfeit goods in place of authentic items.

At event 120, the method 100 further comprises extracting genomic DNA from the collected cotton fibers. In general, a variety of nucleic acid extraction solutions have been developed over the years for extracting nucleic acid sequences from a sample of interest. See, for example, Sambrook et al. (Eds.) Molecular Cloning, Cold Spring Harbor Press, 1999. Many such methods typically require, for example, a detergent-mediated step, a proteinase treatment step, a phenol and/or chloroform extraction step, and/or an alcohol precipitation step. Some nucleic acid extraction solutions may comprise an ethylene glycol-type reagent or an ethylene glycol derivative to increase the efficiency of nucleic acid extraction while other methods use only grinding and/or boiling the sample in water. Other methods, including solvent-based systems and sonication, could also be utilized in conjunction with other extraction methods.

In most embodiments, about 5 mg to about 30 mg of cotton fiber or cotton lint, and in certain instances between about 10 mg to about 15 mg of cotton fibers are needed for sufficient DNA to be extracted from the sample for analysis. DNA extraction protocols are derived from standard molecular biology DNA extraction procedures, which can be easily accomplished by people skilled in the art.

While extracting DNA from seeds and leaves from various cotton species is quite common for cotton research genomics, utilizing DNA from the mature cotton fibers is quite unusual and unique. The amount of DNA as well as the integrity of the DNA isolated from mature cotton fibers may not be adequate for researching genetic variability and relationships among a plurality of genotypes or cotton cultivars. The use of cotton fibers as a DNA source as described herein is, surprisingly, sufficient for identifying and distinguishing between a plurality of different cotton cultivars. This is partly due to the simplicity of the genetic test preformed in the methods.

The embodiment of FIG. 2, in event 130, further comprises amplifying a target sequence or specific DNA region of the cotton DNA genome extracted from the cotton fibers in event 120. Polymerase Chain Reaction procedures enables the target sequence within the cotton cultivar genome to be amplified, or copied, to a detectable quantity. In general, amplification comprises temperature cycling of the genomic DNA sample in the presence of free nucleotides, a specific set of primers for the target DNA, along with a polymerase which allows the target DNA to be copied, i.e. amplified.

Utilizing primer software known to those skilled in the art, a plurality of primers were identified and designed for the selected variable region(s) of the cotton cultivar DNA sequences, allowing for the amplified PCR product(s) to be identified as being from a particular cultivar of particular Gossypium cotton species. PCR primers were designed according to known criteria and PCR procedures may be conducted in commercially available instruments. Primers were designed to distinguish between the targeted sequences of the cultivars by exploiting the differences in at least one physical characteristic related to the targeted sequences. The “physical characteristic” utilized to distinguish between the cotton cultivars is either a detectable difference in length/size of the targeted sequence or a detectable DNA sequence variation (i.e. base substitution) specific for each of cotton cultivars being analyzed.

Generally, the primers designed for PCR amplification in event 130 utilize simple sequence repeats (SSR) or microsatellites which are polymorphic loci present in nuclear and organellar DNA (i.e. chloroplast DNA). Identifying regions comprising SSRs enables one set of primers to amplify a target sequence in a plurality of cultivars of a particular Gossypium cotton species, with the target sequences from one cultivar to another being different in length due the presence of varying number of SSRs found in the target sequence.

In most embodiments, when a large number of cultivars need to be distinguished from one another, more than one primer set is utilized in the methods. The number of primer sets needed to identify a particular cultivar is dependent on the number of possible cultivars that the sample may be from. When there are only two cultivar possibilities for the sample, one primer set may be all that is needed to distinguish between the two cultivars. When there are a multitude of possible cultivars that the sample may have originated from, about two to about seven primer sets may be utilized to analyze the cotton fiber sample. In Example 1 below, one primer set was utilized to distinguish between the genotypes of cultivars of G. barbadense. In some embodiments, the range of primers set utilized are from about one primer set to about ten primer sets, more likely from about two primer sets to about seven primer sets, and even more likely from about three primer sets to about five primer sets.

In some embodiments, nested set primers are utilized to achieve appropriate amplification of the targeted variable region(s) of the cotton cultivars of interest. Again, these nested set primers were identified and designed utilizing primer software and the genomic sequences of the cotton cultivar of interest using primer design methods known to those skilled in the art of primer design.

The method 100 illustrated in FIG. 2 further comprises analyzing the PCR product(s) for a specific physical characteristic profile related to only one of the cotton cultivars of interest in event 140. When the identifying physical characteristic profile is the length or size of the PCR products produced in event 130, techniques which allow for efficient size differentiation between the PCR products produced by one cotton cultivar over another are utilized.

With capillary electrophoresis, it is possible to distinguish between the varying sizes of the PCR products produced by DNA from mature cotton fibers of G. barbadense cultivars. For example, when utilizing primer set SEQ ID NO: 1 and SEQ ID NO:2 for PCR amplification, the PCR products produced by G. barbadense cultivars can be distinguished by capillary electrophoresis. Utilizing an internal standard of known size in the capillary electrophoresis experiments enables the lengths (e.g. number of base pairs) of the PCR products of each cultivar to be determined and the profiles compared to one another. Thus the two cultivars can be uniquely identified from one another using the methods.

In most embodiments, where the physical characteristic of the PCR products being analyzed in event 140 is size/length, a capillary electrophoresis device such as an AB1310 genetic analyzer can be utilized to determine the size of the PCR product(s) produced. For capillary electrophoresis, the length of the DNA amplicons produced by PCR is determined by extrapolating size data from an internal control run with the sample of known size. This allows for the capillary electrophoresis results of multiple PCR experiments, each with a different set of primers, to be overlaid to provide a variable amplicon length polymorphism (VALP) profile for each cultivar of interest. See example 1 below.

In general, the size resolution of the capillary electrophoresis is capable of distinguishing between amplicons which differ by only about 2 base pairs. The sensitivity of capillary electrophoresis enables in some embodiments the use of primers which target variable regions of the cotton cultivar where the difference between the size of a first cultivar amplicon and a second cultivar amplicon is less than about 10 base pairs but greater than about 2 base pairs, and in some embodiments the size difference can be less than about 6 base pairs but greater than about 3 base pairs. In an even broader embodiment, the size can range from 1 base pair to 60 base pairs.

In event 150, the cultivar of the analyzed cotton fibers is determined. This determination can be made by comparing the identified VALP profile of the PCR products produced by the sample to the theoretical and actual known sizes of the targeted and amplified sequences for specified G. barbadense cultivars in event 150. The VALP profiles of various cultivars may be comprised in a data base or cotton cultivar VALP genotype bank which the VALP results obtained from the sample can be compared to and aid in cultivar identification.

The PCR products from known G. barbadense cultivars may also be run on a gel during electrophoresis to determine a VALP profile for the cotton sample and aid in identifying the cultivar from which the sample originated. Once the lengths of the PCR products has been determined by either gel electrophoresis, capillary electrophoresis or other similar DNA sizing techniques, the analyzed sample can be identified and confirmed as a particular cotton cultivar 150.

In other embodiments, the physical characteristic being analyzed is not length but a DNA sequence specific to a particular cultivar. In this situation, PCR amplification may be performed in the presence of a non-primer detectable probe which specifically binds the PCR amplification product produced by one cultivar and not by other cultivars. PCR primers are designed according to known criteria and PCR may be conducted in commercially available instruments. The probe is preferably a DNA oligonucleotide specifically designed to bind to the amplified PCR product. The probe preferably has a 5′ reporter dye and a downstream 3′ quencher dye covalently bonded to the probe which allows fluorescent resonance energy transfer. Suitable fluorescent reporter dyes include 6-carboxy-fluorescein (FAM), tetrachloro-6-carboxy-fluorescein (TET), 2,7-dimethoxy-4,5-dichloro-6-carboxy-fluorescein (JOE) and hexachloro-6-carboxy-fluorescein (HEX). A suitable reporter dye is 6-carboxy-tetramethyl-rhodamine (TAMRA). These dyes are commercially available from Perkin-Elmer, Philadelphia, Pa. Detection of the PCR amplification product may occur at each PCR amplification cycle. At any given cycle during the PCR amplification, the amount of PCR product is proportional to the initial number of template copies. The number of template copies is detectable by fluorescence of the reporter dye. When the probe is intact, the reporter dye is in proximity to the quencher dye which suppresses the reporter fluorescence. During PCR, the DNA polymerase cleaves the probe in the 5′-3′ direction separating the reporter dye from the quencher dye increasing the fluorescence of the reporter dye which is no longer in proximity to the quencher dye. The increase in fluorescence is measured and is directly proportional to the amplification during PCR. This detection system is now commercially available as the TaqMan® PCR system from Perkin-Elmer, which allows real time PCR detection.

A probe specific for a first G. barbadense cultivar PCR product may be analyzed in the same or a separate PCR sample tube as the probe specific for a second G. barbadense cultivar PCR product. Utilizing real-time PCR, the signal from a probe corresponding to a specific cotton cultivar will identify which cotton cultivar has been collected. It is also possible to attached unique probes for each cultivar to a micro-array and test the sample for a plurality of cultivars utilizing micro-array technology.

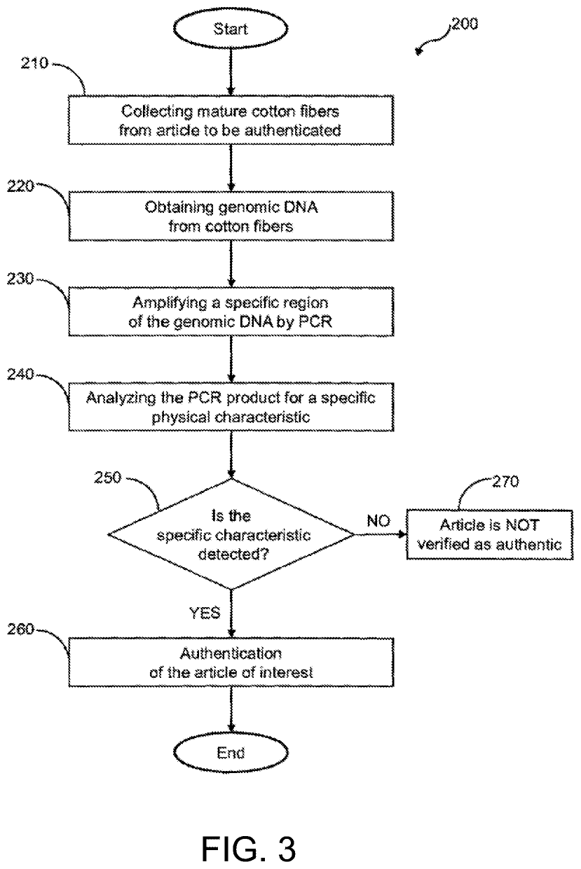

FIG. 3 is a flow chart illustrating generally a method 200 for authenticating or identifying an article as being manufactured with a specified cotton cultivar. Usually verifying or authenticating an article occurs after the article has been introduced into a supply chain. Frequently, counterfeiters and forgers have the best access to articles after they are being shipped from the manufacturer/producer to wholesale or retail outlets or locations. With the methods described herein, a producer can track and authenticate articles or goods, allowing for better monitoring of when and where counterfeit goods are being replaced with forgeries or otherwise altered. When the article has entered a supply chain, a manufacturer or an authorized individual can collect a sample from the article at any desired point along the supply chain for authentication purposes.

The method 200 comprises, at event 210, collecting mature cotton fibers from an article to be authenticated. Collecting a sample may involve scraping, cutting or dissolving a portion of the article, to obtain a cotton fiber sample for analysis. The collecting of the sample may be carried out, for example, by cutting the article to remove mature cotton fibers from the article. The sample collecting in other embodiments may be achieved by tweezing, scraping, or abrading the article with appropriate sampling tools configured to remove a sufficient amount of cotton fibers or cotton lint for analysis. Collecting fiber samples may, for example, occur at any point along the supply or commerce chain where there is concern about or risk of introduction of counterfeit articles.

The method 200 for authenticating an article further comprises, in event 220, obtaining or extracting genomic DNA from the collected cotton fibers. Extraction, isolation or purification of the genomic DNA obtained in the sample may be required. A variety of nucleic acid extraction solutions have been developed over the years for extracting nucleic acid sequences from a sample of interest. See, for example, Sambrook et al. (Eds.) Molecular Cloning, Cold Spring Harbor Press, 1999. Many such methods typically require one or more steps of, for example, a detergent-mediated step, a proteinase treatment step, a phenol and/or chloroform extraction step, and/or an alcohol precipitation step. Some nucleic acid extraction solutions may comprise an ethylene glycol-type reagent or an ethylene glycol derivative to increase the efficiency of nucleic acid extraction while other methods only use grinding and/or boiling the sample in water. Other methods, including solvent-based systems and sonication, could also be utilized in conjunction with other extraction methods.

The authentication method 200 further comprises, in event 230, amplifying at least one specific region of the extracted genomic DNA by PCR based techniques. The procedures and techniques for PCR amplification of a targeted sequence are similar to those described above in the methods illustrated in FIG. 2. There are numerous cultivars of each of the species G. barbadense and G. hirsutum which are utilized in the manufacturing of cotton textiles worldwide. The primers designed and selected in many embodiments enable a user to distinguish between the cultivars of G. barbadense. It should be noted that other cultivars from various cotton species ELS or Upland, and in particular G. hirsutum, can also be identified using the methods described herein. In other embodiments primers may be selected and used for distinguishing cultivars from different cotton species.

The method 200 for authenticating an article further comprises, in event 240, analyzing the PCR product(s) for a specific physical characteristic or specific physical characteristic profile, such as length of the PCR product(s) or a specific DNA sequence associated with a specific cotton cultivar (e.g. G. barbadense). Analyzing the PCR product produced is generally the same as described above in the methods illustrated in FIG. 2. Numerous ELS cultivars have been shown to produce the identical or very similar length PCR products, which are easily identifiable as G. barbadense and are distinguishable from various Upland cultivars of G. hirsutum. In general, to distinguish between cultivars of a particular species, a plurality of primers sets are utilized to obtain a variable amplicon length polymorphism (VALP) profile for each cultivar of interest. Example 1 below the identification or VALP profile results of various cultivars in more detail. When the aim is to authenticate a cotton textile as being a specific G. barbadense cultivar, the analysis of the sample can be simplified to utilize the least number of primer sets which will determine if the samples in question have the associated VALP to the cultivar of interest. In many instances, a textile shipment may be on a stringent time table and a quick but accurate test is required, thus limiting the amount of time to perform the analysis is preferred. Utilizing only those primer sets which are needed to produce a particular cultivar's VALP profile fulfills the desire for a quick and reliable verification test.

In event 250, the results of the analysis of the collected sample are reviewed and a query or determination is made as to whether or not a physical characteristic (i.e. known PCR product length) or the VALP profile for a particular cultivar was detected in the sample. If the physical characteristics associated with a particular cotton cultivar nucleic acid is not found or not detected in the collected sample of the textile article at event 250, the conclusion at event 270 from the analysis is the that article is not made from that particular cotton cultivar and may have been tampered with or substituted. If the physical characteristic and/or VALP profile associated with the cotton cultivar is detected in the sample at event 250, then the article is verified in event 260 as being authentic.

If a determination is made in event 270 that an article is not authentic, a different, earlier point in the supply or commerce chain may be selected and events 210 through 250 may be repeated. Thus an article from an earlier point in the supply chain would be selected, cotton fibers collected, and analyzed. If it is again determined that the article is not authentic or has been otherwise tampered with, then events 210-250 may be repeated with an article selected from yet an earlier point in the supply chain. In this manner, the time and/or location of tampering or counterfeit substitution of the textile article may be located.

The invention also provides kits for verifying cotton fibers and authenticating articles of interest using the methods and kit. The kits may comprise, for example, a container of the nucleic acid extraction buffer, and a sample tube for holding a collected mature cotton fiber sample of the item or article to be authenticated. The kits may include at least one primer set configured to produce amplified PCR fragments from a plurality of cultivars (e.g. cultivars of G. barbadense species), e.g., SEQ ID NO: 1 and SEQ ID NO: 2, defined below. The kits may further comprise a collection tool for taking a sample of the labeled article for transfer to the sample tube. The kits may also include a portable electrophoretic device (e.g. gel apparatus or capillary electrophoresis system) for analyzing PCR products. The kits may still further comprise an internal control for fragment size comparison for capillary analysis.

By way of example, the collection tool of the kit may comprise a blade or scissors, or the like, for cutting a piece of the article. The sample tube of the kit may comprise a sealable vial or Eppendorf tube, and may contain solvent or solution for extraction of the nucleic acids (e.g. DNA) from the sample taken from the textile article.

The kit may further comprise primers and/or probes as well as solutions appropriate for PCR analysis. The kit may further comprise a small PCR instrument for analysis of the extracted nucleic acids from the mature cotton fibers.

The capillary electrophoresis device of the kit may comprise an internal control for detecting the fragment size of the amplified PCR product(s).

In many embodiments, the kit will further comprise a system for accessing a data base of the genotypes (i.e. VALP profiles) of cultivars of interest, for comparison to the results obtained from the cotton fiber sample. The illustrative kits thus provide a convenient, portable system for practicing the methods described herein.

Illustrative methods for authenticating textile articles utilizing mature cotton fibers are provided in the following Example.

### Example 1

The following assay distinguishes Giza94 from other varieties of G. barbadense (e.g., US Pima and non-Giza94 Egyptian cotton (e.g., G86, G92, G45, G70, G87, G88, etc.)). The target of the assay is SSR loci.

The inventors of the present invention have found a marker, BNL 1531, which corresponds to Egyptian cotton. This marker BNL1531 is a length polymorphism. In the non-Giza94 G. barbadense varieties, there are three (3) major alleles (i.e. 3 major peaks in CE traces) at 115 bp, 121 bp and 126 bp. In contrast, in Giza94, there is an extra allele at 128 bp, in addition to the aforementioned three (i.e., in Giza94, four major peaks are shown in CE traces). See FIG. 1.

The target sequence (in G. hirsutum) is below:

>BNL1531 ID=BNL1531|Name=BNL1531|organism=Gossypium hirsutum|type=genetic_marker|length=140 bp

(SEQ ID NO: 3)
CTGCAACAAGAGCCTGTGTCGGAAACTACATGCATGAAGAGAGAGAGAG
AGAGAGAGAGAGAGAGAAAACTTGTTCATATTCCAAGTCAAATTTCAAA
AACTCATCATCGCAGTTCTCTACCATCTCAGCCAATCTCCAT.

The primer sequences are:

BNL1531_F:
(SEQ ID NO: 1)
CTGCAACAAGAGCCTGTGTC
BNL1531_R:
(SEQ ID NO: 2)
ATGGAGATTGGCTGAGATGG

FIG. 1 is a PCR/CE assay showing an extra peak at ˜128 bp in Giza94 (lower panel) that does not exist in Giza86 (upper panel) or in US Pima.

G. barbadense and G. hirsutum share the same flanking primer regions but since there are variations in the middle of the target region, it is possible to distinguish between them. Given that the flanking regions are conservative enough between G. hirsutum and G. barbadense (i.e., share the same flanking sequence), one could modify the primer sequences to amplify a larger or smaller fragment that still covers the target region. The point of having shared primers is to reduce the complexity of the assay, so one can use a single pair of primers to amplify across different species and varieties.

The desired region is amplified by PCR, put on a gel and analyzed by CE.

1. A method for determining whether a mature cotton fiber includes Giza 94 (G94) variety of G. barbadense, the method comprising:
providing a mature cotton fiber from a manufactured article;
extracting DNA from the mature cotton fiber;
amplifying a portion of said DNA with a PCR-based technique using a primer set comprising SEQ ID NO: 1 and SEQ ID NO: 2, thereby producing one or more amplicons,
generating a variable amplicon length polymorphism profile (VALP) from the amplicons, and
comparing the generated VALP with a G94 variable amplicon length polymorphism profile, thereby determining whether the mature cotton fiber incudes G94.

2. The method of claim 1 wherein the DNA comprises nuclear DNA.

3. The method of claim 1, wherein the DNA comprises mitochondrial DNA.

4. The method of claim 1, wherein the DNA comprises chloroplast DNA.

5. The method of claim 1 wherein the generated VALP is obtained by capillary electrophoresis.

6. The method of claim 1 wherein the G94 variable amplicon length polymorphism profile comprises peaks in capillary electrophoresis traces at 115 bp, 121 bp, 126 bp and 128 bp.

7. The method of claim 1 wherein the generated VALP is obtained by gel electrophoresis.

8. The method of claim 1, further comprising quantifying the one or more of the amplicons by real-time PCR.

9. The method of claim 1, further comprising analyzing an additional detectable physical characteristic.

10. The method of claim 9, wherein the additional detectable physical characteristic comprises a difference in nucleotide composition.

11. The method of claim 9 wherein analyzing an additional detectable physical characteristic comprises the use of a hybridizing probe specific for the target sequence of G94.

12. The method of claim 1, wherein the manufactured article comprises raw mature cotton, ginned mature cotton, yarn blended with mature cotton and/or fabric blended with mature cotton.

13. The method of claim 1, wherein said manufactured article is a garment or textile.

14. The method of claim 13 wherein the textile is selected from the group consisting of yarn, fabric, ginned cotton, an artwork canvas and an anthropological textile.

15. A method for verifying authenticity of the variety of G. barbadense of a mature cotton fiber as including a fiber from G94, wherein the mature cotton fiber is from a manufactured article, the method comprising:
providing a mature cotton fiber from the manufactured article;
extracting DNA from the mature cotton fiber;
amplifying a portion of said DNA with a PCR-based technique using a primer set consisting of SEQ ID NO: 1 and SEQ ID NO: 2, thereby producing one or more amplicons,
generating a variable amplicon length polymorphism profile (VALP) from the amplicons, and
comparing the generated VALP with a G94 variable amplicon length polymorphism profile, thereby verifying the authenticity of the mature cotton fiber from the manufactured article as including a fiber from G94.

16. A kit for determining whether a mature cotton fiber includes Giza 94 (G94) variety of G. barbadense, the kit comprising at least one primer set comprising SEQ ID NO: 1 and SEQ ID NO: 2.

17. The kit of claim 16 further comprising a collection tool for taking a sample of the mature cotton fiber for transfer to a sample tube and electrophoretic device for analyzing PCR products.

18. The kit of claim 17, wherein the electrophoretic device is a gel apparatus or capillary electrophoresis system.


SEQUENCE LISTING (3 sequences)

SEQ ID NO: 1 - DNA, 20 bp, synthetic construct
  misc_feature 1..20: note = Description of sequence: Primer: BNL1531_F
  source 1..20: mol_type = other DNA
ctgcaacaagagcctgtgtc

SEQ ID NO: 2 - DNA, 20 bp, synthetic construct
  misc_feature 1..20: note = Description of sequence: Primer: BNL1531_R
  source 1..20: mol_type = other DNA
atggagattggctgagatgg

SEQ ID NO: 3 - DNA, 140 bp, Gossypium hirsutum
  source 1..140: mol_type = genomic DNA
ctgcaacaagagcctgtgtcggaaactacatgcatgaagagagagagagagagagagagagagagaaaacttgttcatattccaagtcaaatttcaaaaactcatcatcgcagttctctaccatctcagccaatctccat